(19) United States
(12) Patent Application Publication
(10) Pub. No.: US 2020/0022797 A1
(43) Pub. Date: Jan. 23, 2020

(54) IVC FILTER RETRIEVAL KIT

(71) Applicant: Mohammad Reza Rajebi, Denver, CO (US)

(72) Inventor: Mohammad Reza Rajebi, Denver, CO (US)

(21) Appl. No.: 15/978,056

(22) Filed: May 11, 2018

Related U.S. Application Data
(60) Provisional application No. 62/582,722, filed on Nov. 7, 2017.

(51) Int. Cl. A61F 2/01

(52) U.S. Cl. CPC A61F 2/01; A61B 2017/22035; A61F 2002/016; A61F 2002/011

(57) ABSTRACT

A surgical kit used for removing a vascular filter, particularly an Inferior Vena Cava (IVC) filter. The surgical kit includes a forceps-tool and a sheath. The forceps-tool includes a pair of interlocking-jaw members configured to grasp a hook of the vascular filter in a clamped-position. Once clamped, the sheath can be extended over a body of the vascular filter to retrieve and remove the vascular filter.

### CROSS REFERENCE TO RELATED APPLICATION

The present application is related to and claims priority to U.S. Provisional Patent Application No. 62/582,722 filed Nov. 7, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of vascular filters and more specifically relates to a kit for removing vascular filters.

### 2. Description of Related Art

An IVC filter is a metal device that is positioned into the body's main vein—called the inferior vena cava (IVC)—that leads from the legs to the right side of the heart. This filter can help keep clots from being carried into the lungs. Doctors may leave the IVC filters in place, causing long-term complications for patients. Patients may travel to specific hospitals that have experience in retrieving difficult filters; however, this can be expensive and impossible for some. Further, doctors may use tools not specifically-designed for removing IVC filters and cause additional injury. An effective alternative is needed.

U.S. Pat. No. 6,726,621 to Naroun Suon relates to retrieval devices for vena cava filter. The described retrieval devices for vena cava filter includes a shaft having a proximal end, a distal end, and a lumen extending therethrough, a wire having a first end and a second end, the wire being partially disposed within the lumen of the shaft, a portion of the wire extending beyond the distal end of the shaft and forming a loop, and a portion of the wire extending beyond the proximal end of the shaft.

### BRIEF SUMMARY OF THE INVENTION

In view of the foregoing disadvantages inherent in the known vascular filters art, the present disclosure provides a novel IVC filter retrieval kit. The general purpose of the present disclosure, which will be described subsequently in greater detail, is to provide a kit for effectively and safely removing an Inferior Vena Cava (IVC) filter.

A surgical kit is disclosed herein. The surgical kit includes a forceps-tool may include a handle-end, a clamping-end opposite the handle-end, and an elongate-shaft therebetween. The clamping-end may have a pair of interlocking-jaw members movable between a clamped-position and an unclamped-position via manipulation of the handle-end. The interlocking-jaw members may be configured to grasp the hook of the vascular filter in the clamped-position.

Further, a sheath may be provided and may include a first open-end, a second-open end opposite the first open-end and an elongate hollow-body therebetween. The sheath may be configured for placement over the elongate-shaft of the forceps-tool such that the elongate-shaft may be located within the elongate hollow-body of the sheath. In addition, the sheath may be freely moveable along the elongate-shaft into an extended-position and configured to envelope the body of the vascular filter when in the extended-position.

A method of using the surgical kit is also disclosed herein. The method of using surgical kit may comprise the steps of: providing the surgical kit as above; placing the sheath over the elongate-shaft of the forceps-tool; manipulating the interlocking-jaw members via the handle-end of the forceps-tool; grasping the hook of the vascular filter with the interlocking-jaw members in the clamped-position; locking the interlocking-jaw members in the clamped-position; moving the elongate hollow-body into the extended-position; enveloping the body of the vascular filter with the elongate hollow-body in the extended-position; capturing the vascular filter; and removing the vascular filter.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, an IVC filter retrieval kit, constructed and operative according to the teachings of the present disclosure.

FIG. 1 is a front perspective view of the surgical kit during an ‘in-use’ condition, according to an embodiment of the disclosure.

FIG. 2A is a front-side perspective view of the surgical kit of FIG. 1, according to an embodiment of the present disclosure.

FIG. 2B is a front-side perspective view of the surgical kit of FIG. 1, according to an embodiment of the present disclosure.

FIG. 3A is a front-side perspective view of the surgical kit of FIG. 1, according to an embodiment of the present disclosure.

FIG. 3B is a front-side perspective view of the surgical kit of FIG. 1, according to an embodiment of the present disclosure.

FIG. 4 is a front perspective view of the surgical kit of FIG. 1, according to an embodiment of the present disclosure.

FIG. 5 is a front perspective view of the surgical kit of FIG. 1, according to an embodiment of the present disclosure.

FIG. 6 is a flow diagram illustrating a method of use for the disclosed surgical kit, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to vascular filters and more particularly to an IVC filter retrieval kit as used to improve the kit for removing vascular filters.

Generally, disclosed is a kit used to remove indwelled Inferior Vena Cava (IVC) filters which may be typically difficult to retrieve with snares. The kit may feature a pair of jaw mechanisms on one end of the tool capable of grasping the hook on the top of the filter. Further, a sheath or tube may be provided to fit over the forceps. The forceps and the sheath may include a smooth bend in shape which can be extended over the IVC filter once grasped within the artery. This may enable medical personnel to retrieve filters embedded into artery sidewalls. The kit may eliminate the worry of patients suffering from long-term complications such as IVC thrombosis.

As above, the IVC Filter Retrieval Kit may include a pair of stainless steel forceps designed for IVC filter removal equipped with a vascular sheath. The vascular sheath and forceps may feature a bend at a distal end. A tip of the sheath may be reinforced with a metallic ring. The forceps may include two handles and two interlocking jaws. The jaws may be used to grip and remove the filter from an artery wall. Once the filter is grabbed, a locking mechanism may be actuated to hold the apex of the filter. The vascular sheath may then be extended over the filter in order to capture it. The kit may be intended to safely and securely remove the filter to prevent further complications such as IVC thrombosis.

The IVC Filter Retrieval Kit may be constructed using stainless steel, plastic, and other suitable materials. The device may be available in a universal size capable of accommodating all user needs and preferences. Exact size, measurement, construction, and design specifications may vary upon manufacturing.

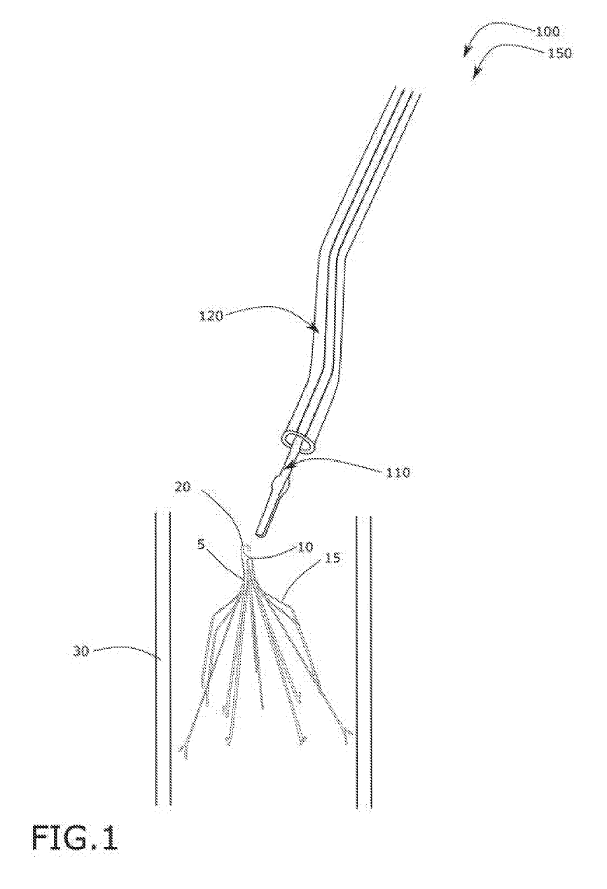

Referring now more specifically to the drawings by numerals of reference, there is shown in FIGS. 1-5, various views of a surgical kit 100.

FIG. 1 shows a surgical kit 100 during an ‘in-use’ condition 150, according to an embodiment of the present disclosure. As illustrated, the surgical kit 100 may include a forceps-tool 110 and a sheath 120. The surgical kit 100 may be used for removing a vascular filter 5. Here, the vascular filter 5 is shown to be an Inferior Vena Cava (IVC) filter. The vascular filter 5 may include a top 10 and a body 15. As shown, the top 10 may have a hook 20 extending therefrom.

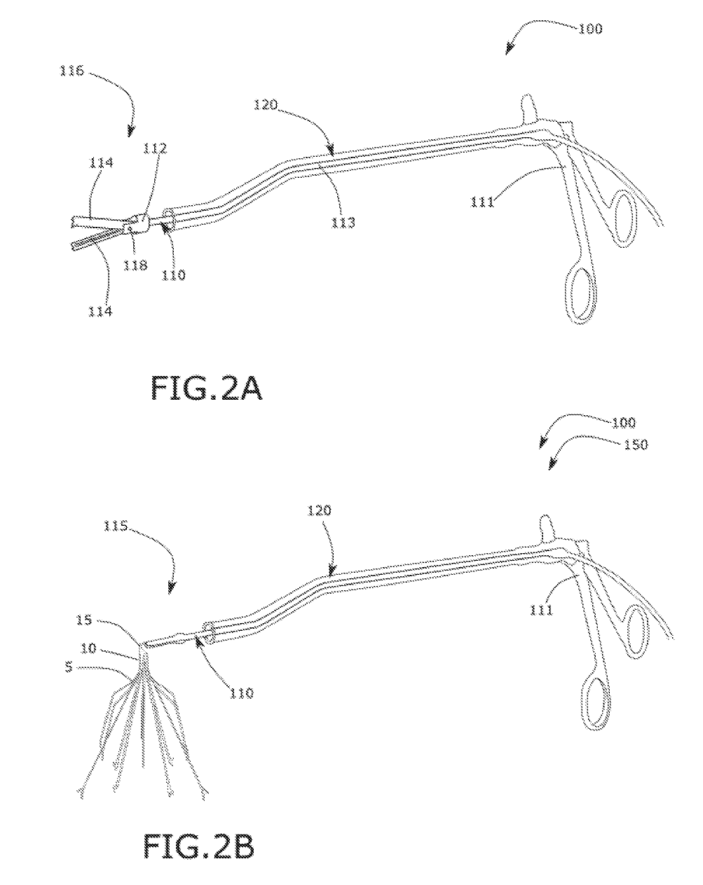

FIG. 2A-2B show side-front perspective views of the surgical kit 100 of FIG. 1, according to an embodiment of the present disclosure. Shown in FIG. 2B is the forceps-tool 110 grasping the top 10 of the vascular filter 5. The forceps-tool 110 may include a handle-end 111, a clamping-end 112 opposite the handle-end 111, and an elongate-shaft 113 therebetween. The forceps-tool 110 should be biocompatible. In a preferred embodiment, the forceps-tool 110 may be made from a material configured to withstand repeated sterilization. In one example, the material may be stainless steel. In another example, the material may be carbon steel. However, it should be appreciated that other materials may be used, including any advancements in sterilized materials not currently known. Further, the forceps-tool 110 may be disposable in some embodiments, and re-usable in other embodiments.

As shown, the clamping-end 112 may include a pair of interlocking-jaw members 114 movable between a clamped-position 115 and an unclamped-position 116 via manipulation of the handle-end 111. The interlocking-jaw members 114 may be configured to grasp the hook 20 of the vascular filter 5 in the clamped-position 115. Further, the clamping-end 112 may include a locking-mechanism 118 configured to lock the interlocking-jaw members 114 in the clamped-position 115. In one embodiment, the locking-mechanism 118 may be actuated manually via an actuation-means such as a button, switch, etc. In another embodiment, the locking-mechanism 118 may be automatically actuated.

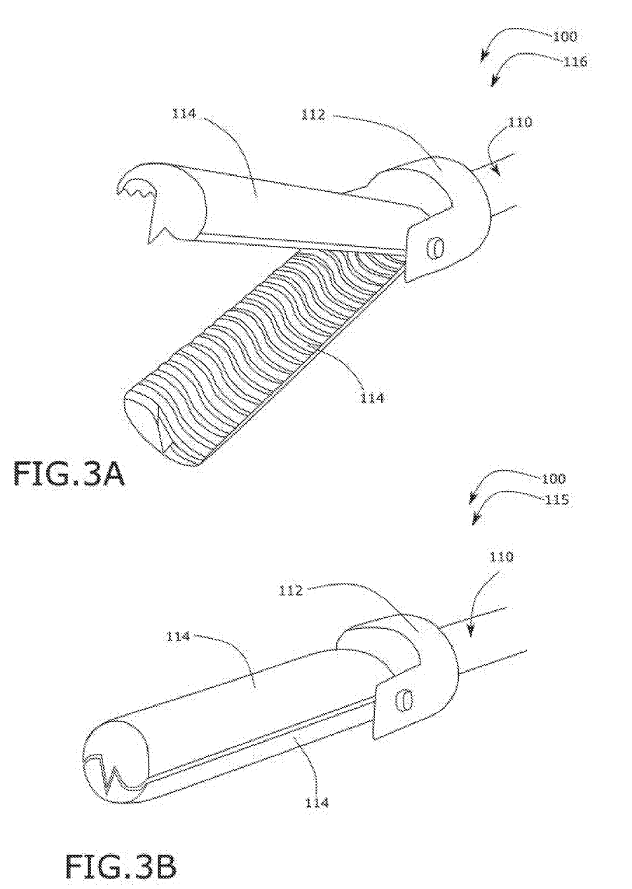

FIGS. 3A-3B show a front-side view of the surgical kit 100 of FIG. 1. Shown here is a close-up view of the clamping-end 112 of the forceps-tool 110. As above, the clamping-end 112 may include the interlocking-jaw members 114. Here, they are shown to be in the unclamped-position 116 (FIG. 3A) and the clamped-position (FIG. 3B). These figures illustrate the unique shape of the interlocking-jaw members 114 whereby a top interlocking-jaw member and a bottom interlocking-jaw member form a ‘yin and yang’ style shape when in the clamped-position 115. Further, the interlocking-jaw members may include an oval shape. This configuration may be useful for when the hook 20 of the vascular filter 5 is embedded into a wall of the IVC (FIG. 1). An edge of the interlocking-jaw members 114 may detach the hook 20 before capturing it (FIG. 1).

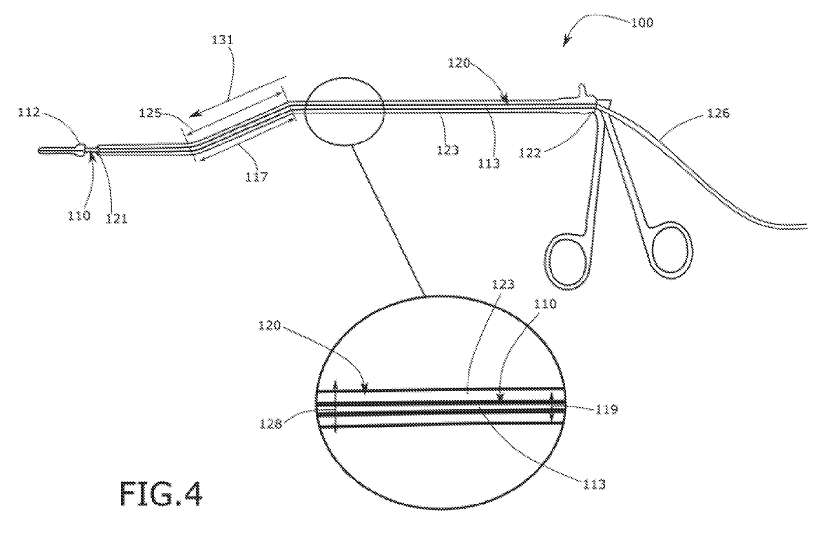

Referring now to FIG. 4 showing a side perspective view of the surgical kit 100 of FIG. 1, according to an embodiment of the present disclosure. Shown in this figure is the functional relationship between the forceps-tool 110 and the sheath 120. The sheath 120 may include a first open-end 121, a second open-end 122 opposite the first open-end 121 and an elongate hollow-body 123 therebetween. As shown, the sheath 120 may be configured for placement over the elongate-shaft 113 of the forceps-tool 110 such that the elongate-shaft 113 is located within the elongate hollow-body 123 of the sheath 120.

The elongate-shaft 113 may include a first substantially bent-section 117 about the clamping-end 112 of the forceps-tool 110. Similarly, the elongate hollow-body 123 may include a second substantially bent-section 125 about the first open-end 121 and configured to conform to the first substantially bent-section 117 of the elongate-shaft 113. As shown, the first substantially bent-section 117 and the second substantially bent-section 125 may include a downward-angle 131. The downward-angle 131 may provide improved precision when performing the surgical procedure.

Further to this, the sheath 120 may be freely moveable along the elongate-shaft 113 into an extended-position 124 (FIG. 4). To aid in this, the elongate hollow-body 123 may include a sheath-diameter 128 at least double a forceps-diameter 119 of the elongate-shaft 113 configured to provide free movability of the sheath 120 over the elongate-shaft 113. In other embodiments, the sheath-diameter 128 may be more or less than double the forceps-diameter 119. In addition, the sheath 120 may further include a tubing 126 extending about the second open-end 122 and configured to push the sheath 120 into the extended-position 124 (FIG. 4). The tubing 126 may allow a surgeon to push the sheath 120 into the extended-position 124 (FIG. 4).

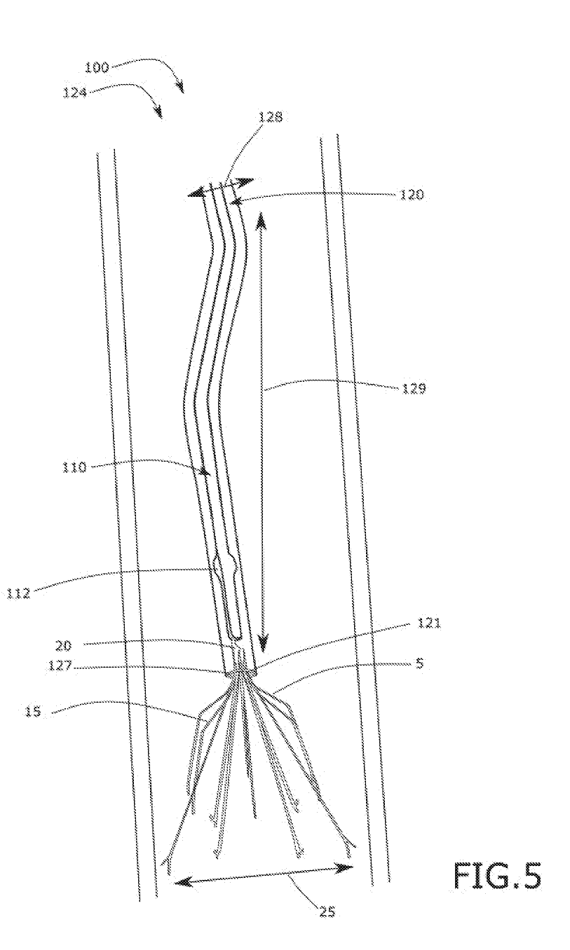

FIG. 5 shows a front perspective view of the surgical kit 100 of FIG. 1, according to an embodiment of the present disclosure. Shown here is the sheath 120 during retrieval of the vascular filter 5. As above, the sheath 120 may be freely moveable along the elongate-shaft 113 into the extended-position 124. In this embodiment, the sheath 120 may be configured to envelope the body 15 of the vascular filter 5 when in the extended-position 124. As shown, the extended-position 124 may include the first open-end 121 being pushed over the clamping-end 112 of the forceps-tool 110 and onto the body of the vascular filter 5 to allow the sheath 120 to envelope the vascular filter 5.

In the preferred embodiment, the first open-end 121 of the sheath 120 may include a reinforcement-ring 127 to provide stability to the open-end of the sheath 120 to prevent sagging when enveloping the vascular filter 5. In this preferred embodiment, the reinforcement-ring 127 may be made from a metal material. Further, the elongate hollow-body 123 may be made from a flexible-material to allow the sheath 120 to flex over the body 15 of the vascular filter 5. In one embodiment, the flexible-material may be a biocompatible plastic-material. In another embodiment, the flexible-material may be biocompatible silicone. Other materials are also contemplated. In some embodiments, the sheath 120 may be disposable. In other embodiments, the sheath 120 may be re-usable and made from a material configured to withstand repeated sterilization.

In one embodiment, the body 15 of the vascular filter 5 may include a width 25 of between 5 mm-12 mm and the elongate hollow-body 123 may be sized to envelope the body 15 of the vascular filter 5. In one embodiment, the elongate hollow-body 123 may include the sheath diameter 128 of at least 4 mm. Further, in one embodiment, the elongate hollow-body 123 may include a sheath-length 129 of at least 25 mm. It should be appreciated that the stated measurements are not meant to be limiting and different sizes and shapes are contemplated.

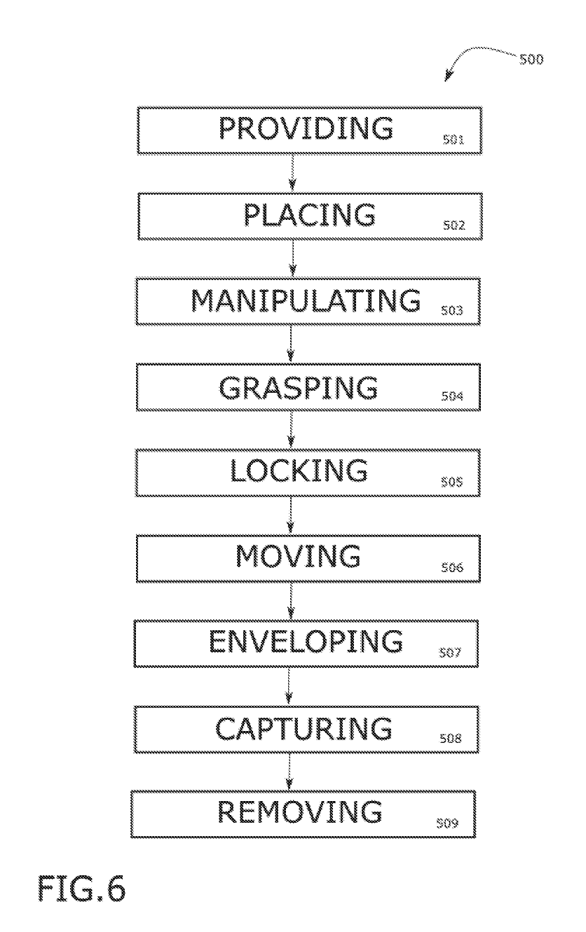

Referring now to FIG. 6, a flow diagram illustrating a method of using a surgical kit for removing a vascular filter 500, according to an embodiment of the present disclosure. As illustrated, the method of using a surgical kit for removing a vascular filter 500 may include the steps of: step one 501, providing the surgical kit 100 as above; step two 502, placing the sheath 120 over the elongate-shaft 113 of the forceps-tool 110; step three 503, manipulating the interlocking-jaw members 114 via the handle-end 111 of the forceps-tool 110; step four 504, grasping the hook 20 of the vascular filter 5 with the interlocking-jaw members 114 in the clamped-position 115; step five 505, locking the interlocking-jaw members 114 in the clamped-position 115; step six 506, moving the elongate hollow-body 123 into the extended-position 124; step seven 507, enveloping the body 15 of the vascular filter 5 with the elongate hollow-body 123 in the extended-position 124; step eight 508, capturing the vascular filter 5; and step nine 509, removing the vascular filter 5.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for surgical kit 100 (e.g., different step orders within above-mentioned list, elimination or addition of certain steps, including or excluding certain maintenance steps, etc.), are taught herein.

Those with ordinary skill in the art will now appreciate that upon reading this specification and by their understanding the art of vascular filters as described herein, methods of vascular filter retrieval will be understood by those knowledgeable in such art.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

1. A surgical kit for removing a vascular filter, the vascular filter including a top and a body, the top having a hook extending therefrom, the surgical kit comprising:
a forceps-tool including
a handle-end,
a clamping-end opposite the handle-end, the clamping-end itself having
a first-jaw having a first-concave-grip, a first-convex-grip, and a tooth,
a second-jaw opposite to and hinged to the first-jaw, the second-jaw having a second-concave-grip conforming to the shape of the first-convex-grip, a second-convex-grip conforming to the shape of the first-concave-grip, and a tooth-relief conforming to the shape of the tooth, and
such that the first-jaw and the second-jaw index to each other with the tooth resting within and filling the tooth-relief, and the first-jaw and the second-jaw when closed together form a cylindrical shape, and
an elongate-shaft therebetween,
wherein the first-jaw and the second-jaw are positionable in a clamped-position and an unclamped-position via manipulation of the handle-end, and wherein the first-jaw and the second-jaw are configured to grasp the hook of the vascular filter in the clamped-position; and
a sheath including
a first open-end,
a second-open end opposite the first open-end and
an elongate hollow-body therebetween, the sheath being configured for placement over the elongate-shaft of the forceps-tool such that the elongate-shaft is located within the elongate hollow-body of the sheath, wherein the sheath is freely moveable along the elongate-shaft into an extended-position, and wherein the sheath is configured to envelope the body of the vascular filter when in the extended-position.

2. The surgical kit of claim 1, wherein the elongate-shaft includes a first substantially bent-section about the clamping-end of the forceps-tool.

3. The surgical kit of claim 2, wherein the elongate hollow-body includes a second substantially bent-section about the first open-end, wherein the second substantially bent-section is configured to conform to the first substantially bent-section of the elongate-shaft.

4. The surgical kit of claim 3, wherein the first substantially bent-section and the second substantially bent-section include a downward-angle.

5. The surgical kit of claim 1, wherein the clamping-end includes a lock configured to lock the first-jaw and the second-jaw in the clamped-position.

6. The surgical kit of claim 1, wherein the sheath further includes a tubing extending about the second open-end and configured to push the sheath into the extended-position.

7. The surgical kit of claim 6, wherein the extended-position includes the first open-end being pushed over the clamping-end of the forceps-tool and onto the body of the vascular filter.

8. The surgical kit of claim 1, wherein the first open-end of the sheath includes a reinforcement-ring.

9. The surgical kit of claim 8, wherein the reinforcement-ring is made from a metal material.

10. The surgical kit of claim 1, wherein the forceps-tool is made from a material configured to withstand repeated sterilization.

11. The surgical kit of claim 10, wherein the material is stainless steel.

12. The surgical kit of claim 10, wherein the material is carbon steel.

13. The surgical kit of claim 1, wherein the elongate hollow-body is made from a flexible-material.

14. The surgical kit of claim 1, wherein the flexible-material is a plastic-material.

15. The surgical kit of claim 1, wherein the elongate hollow-body includes a sheath-diameter at least double a forceps-diameter of the elongate-shaft configured to provide free movability of the sheath over the elongate-shaft.

16. The surgical kit of claim 15, wherein the elongate hollow-body includes the sheath-diameter of at least 4 mm.

17. The surgical kit of claim 1, wherein the body of the vascular filter includes a width of between 5 mm-12 mm and wherein the elongate hollow-body is sized to envelope the body of the vascular filter.

18. The surgical kit of claim 1, wherein the elongate hollow-body includes a sheath-length of at least 25 mm.

19. A surgical kit for removing a vascular filter, the vascular filter including a top and a body, the top having a hook extending therefrom, the surgical kit comprising:
a forceps-tool including
a handle-end,
a clamping-end opposite the handle-end, the clamping-end itself having
a first-jaw having a first-concave-grip, a first-convex-grip, and a tooth
a second-jaw opposite to and hinged to the first-jaw, the second-jaw having a second-concave-grip conforming to the shape of the first-convex-grip, a second-convex-grip conforming to the shape of the first-concave-grip, and a tooth-relief conforming to the shape of the tooth, and
such that the first-jaw and the second-jaw index to each other with the tooth resting within and filling the tooth-relief, and the first-jaw and the second-jaw when closed together form a cylindrical shape, and
an elongate-shaft therebetween,
wherein the first-jaw and the second-jaw are positionable in a clamped-position and an unclamped-position via manipulation of the handle-end, and wherein the first-jaw and the second-jaw are configured to grasp the hook of the vascular filter in the clamped-position; and
a sheath including
a first open-end,
a second-open end opposite the first open-end and
an elongate hollow-body therebetween, the sheath being configured for placement over the elongate-shaft of the forceps-tool such that the elongate-shaft is located within the elongate hollow-body of the sheath, wherein the sheath is freely moveable along the elongate-shaft into an extended-position, and wherein the sheath is configured to envelope the body of the vascular filter when in the extended-position; and
wherein the elongate-shaft includes a first substantially bent-section about the clamping-end of the forceps-tool;
wherein the elongate hollow-body includes a second substantially bent-section about the first open-end and configured to conform to the first substantially bent-section of the elongate-shaft;
wherein the first substantially bent-section and the second substantially bent-section include a downward-angle;
wherein the clamping-end includes a locking-mechanism configured to lock the first-jaw and the second-jaw in the clamped-position;
wherein the sheath further includes a tubing extending about the second open-end and configured to push the sheath into the extended-position;
wherein the extended-position includes the first open-end being pushed over the clamping-end of the forceps-tool and onto the body of the vascular filter;
herein the first open-end of the sheath includes a reinforcement-ring;
wherein the reinforcement-ring is made from a metal material;
wherein the forceps-tool is made from a material configured to withstand repeated sterilization;
wherein the material is stainless steel;
wherein the elongate hollow-body is made from a flexible-material;
wherein the elongate hollow-body includes a sheath-diameter at least double a forceps-diameter of the elongate-shaft configured to provide free movability of the sheath over the elongate-shaft;
wherein the elongate hollow-body includes the sheath diameter of at least 4 mm;
wherein the body of the vascular filter includes a width of between 5 mm-12 mm and wherein the elongate hollow-body is sized to envelope the body of the vascular filter; and
wherein the elongate hollow-body includes a sheath-length of at least 25 mm.

20. (canceled)
